SEQUENCE LISTING (58 sequences)

SEQ ID NO: 1 - DNA, 38 bp, Artificial Sequence
  Synthesized in laboratory.  Not extracted from any organism.
gaaggtcggagtcaacggattctccccagttcgctcca

SEQ ID NO: 2 - DNA, 21 bp, Artificial Sequence
  Synthesized in laboratory.  Not extracted from any organism.
gaaggtcggagtcaacggatt

SEQ ID NO: 3 - DNA, 17 bp, Artificial Sequence
  Synthesized in laboratory.  Not extracted from any organism.
ctccccagttcgctcca

SEQ ID NO: 4 - DNA, 36 bp, Artificial Sequence
  Synthesized in laboratory.  Not extracted from any organism.
atcggtagcatcgctgaaggtcggagtcaacggatt

SEQ ID NO: 5 - DNA, 21 bp, Artificial Sequence
  Synthesized in laboratory.  Not extracted from any organism.
gaaggtcggagtcaacggatt

SEQ ID NO: 6 - DNA, 15 bp, Artificial Sequence
  Synthesized in laboratory.  Not extracted from any organism.
agcgatgctaccgat

SEQ ID NO: 7 - DNA, 25 bp, Artificial Sequence
  Synthesized in laboratory.  Not extracted from any organism.
aacgtgagtgctagcggagtcttaa

SEQ ID NO: 8 - DNA, 44 bp, Artificial Sequence
  Synthesized in laboratory.  Not extracted from any organism.
gaaggtgaccaagttcatgctcggaattcctcgagtctactagg

SEQ ID NO: 9 - DNA, 39 bp, Artificial Sequence
  Synthesized in laboratory.  Not extracted from any organism.
gaaggtgaccaagttcatgctcgcacgccctaggaaacc

SEQ ID NO: 10 - DNA, 42 bp, Artificial Sequence
  Synthesized in laboratory.  Not extracted from any organism.
gaaggtgaccaagttcatgctagcagctcccactcgagcttt

SEQ ID NO: 11 - DNA, 42 bp, Artificial Sequence
  Synthesized in laboratory.  Not extracted from any organism.
gaaggtgaccaagttcatgctcagctcccactcgagaagtac

SEQ ID NO: 12 - DNA, 38 bp, Artificial Sequence; identical to SEQ ID NO: 1
  Synthesized in laboratory.  Not extracted from any organism.

SEQ ID NO: 13 - DNA, 43 bp, Artificial Sequence
  Synthesized in laboratory.  Not extracted from any organism.
gcgtcttctgtcccatgcgttctttttgtcaagaccgacctgt

SEQ ID NO: 14 - DNA, 21 bp, Artificial Sequence
  Synthesized in laboratory.  Not extracted from any organism.
ggccagggctgggacacaaaa

SEQ ID NO: 15 - DNA, 21 bp, Artificial Sequence
  Synthesized in laboratory.  Not extracted from any organism.
ggacagagcccacccaggcta

SEQ ID NO: 16 - DNA, 26 bp, Artificial Sequence
  Synthesized in laboratory.  Not extracted from any organism.
cctgtacccagatatgtccttcagta

SEQ ID NO: 17 - DNA, 25 bp, Artificial Sequence
  Synthesized in laboratory.  Not extracted from any organism.
cacctgacgcgaagttcctatactt

SEQ ID NO: 18 - DNA, 25 bp, Artificial Sequence
  Synthesized in laboratory.  Not extracted from any organism.
aacgtgagtgctagcggagtcttaa

SEQ ID NO: 19 - DNA, 22 bp, Artificial Sequence
  Synthesized in laboratory.  Not extracted from any organism.
gctgcctcgtcctgcagttcat

SEQ ID NO: 20 - DNA, 36 bp, Artificial Sequence
  Synthesized in laboratory.  Not extracted from any organism.
gtgtgctagcgtcctgaaggtgaccaagttcatgct

SEQ ID NO: 21 - DNA, 15 bp, Artificial Sequence
  Synthesized in laboratory.  Not extracted from any organism.
aggacgctagcacac

SEQ ID NO: 22 - DNA, 36 bp, Artificial Sequence; identical to SEQ ID NO: 4
  Synthesized in laboratory.  Not extracted from any organism.

SEQ ID NO: 23 - DNA, 15 bp, Artificial Sequence
  Synthesized in laboratory.  Not extracted from any organism.
agcgatgctaccgat

SEQ ID NO: 24 - DNA, 32 bp, Artificial Sequence
  Synthesized in laboratory.  Not extracted from any organism.
ggccgactcactgcgcgtcttctgtcccatgc

SEQ ID NO: 25 - DNA, 14 bp, Artificial Sequence
  Synthesized in laboratory.  Not extracted from any organism.
gcagtgagtcggcc

SEQ ID NO: 26 - DNA, 45 bp, Artificial Sequence
  Synthesized in laboratory.  Not extracted from any organism.
gaaggtgaccaagttcatgctcgacatgactcaggatatgaagtt

SEQ ID NO: 27 - DNA, 26 bp, Artificial Sequence
  Synthesized in laboratory.  Not extracted from any organism.
cccacatcttctgcaaagaacaccaa

SEQ ID NO: 28 - DNA, 36 bp, Artificial Sequence; identical to SEQ ID NO: 20
  Synthesized in laboratory.  Not extracted from any organism.

SEQ ID NO: 29 - DNA, 15 bp, Artificial Sequence
  Synthesized in laboratory.  Not extracted from any organism.
aggacgctagcacac

SEQ ID NO: 30 - DNA, 45 bp, Artificial Sequence; identical to SEQ ID NO: 26
  Synthesized in laboratory.  Not extracted from any organism.

SEQ ID NO: 31 - DNA, 26 bp, Artificial Sequence
  Synthesized in laboratory.  Not extracted from any organism.
cccacatcttctgcaaagaacaccaa

SEQ ID NO: 32 - DNA, 36 bp, Artificial Sequence; identical to SEQ ID NO: 20
  Produced by Biosearch Technologies, Incorp., Novato, CA.  Not extracted from any organism.

SEQ ID NO: 33 - DNA, 15 bp, Artificial Sequence
  Created by Biosearch Technologies, Incorp., Novato, CA. Not extracted from any organism.
aggacgctagcacac

SEQ ID NO: 34 - DNA, 38 bp, Artificial Sequence; identical to SEQ ID NO: 1
  Synthesized in laboratory.  Not extracted from any organism.

SEQ ID NO: 35 - DNA, 25 bp, Artificial Sequence
  Synthesized in laboratory.  Not extracted from any organism.
aacgtgagtgctagcggagtcttaa

SEQ ID NO: 36 - DNA, 36 bp, Artificial Sequence; identical to SEQ ID NO: 4
  Produced by Biosearch Technologies Incorp., Novato, CA.  Not extracted from any organism.

SEQ ID NO: 37 - DNA, 14 bp, Artificial Sequence
  Produced by Biosearch Technologies Incorp., Novato, CA.  Not extracted from any organism.
agcgatgctaccga

SEQ ID NO: 38 - DNA, 42 bp, Artificial Sequence
  Synthesized in laboratory.  Not extracted from any organism.
gaaggtgaccaagttcatgctgtccaccttccagcagatgtg

SEQ ID NO: 39 - DNA, 20 bp, Artificial Sequence
  Synthesized in laboratory.  Not extracted from any organism.
ggaggggccggactcgtcat

SEQ ID NO: 40 - DNA, 36 bp, Artificial Sequence; identical to SEQ ID NO: 20
  Synthesized in laboratory.  Not extracted from any organism.

SEQ ID NO: 41 - DNA, 15 bp, Artificial Sequence
  Synthesized in laboratory.  Not extracted from any organism.
aggacgctagcacac

SEQ ID NO: 42 - DNA, 46 bp, Artificial Sequence
  Synthesized in laboratory.  Not extracted from any organism.
gaaggtgaccaagttcatgctgttctttttgtcaagaccgacctgt

SEQ ID NO: 43 - DNA, 22 bp, Artificial Sequence
  Synthesized in laboratory.  Not extracted from any organism.
gctgcctcgtcctgcagttcat

SEQ ID NO: 44 - DNA, 44 bp, Artificial Sequence
  Synthesized in laboratory.  Not extracted from any organism.
gaaggtgaccaagttcatgctcactttggtgggtaaaagaaggc

SEQ ID NO: 45 - DNA, 45 bp, Artificial Sequence
  Synthesized in laboratory.  Not extracted from any organism.
gaaggtcggagtcaacggattccactttggtgggtaaaagaaggt

SEQ ID NO: 46 - DNA, 25 bp, Artificial Sequence
  Synthesized in laboratory.  Not extracted from any organism.
gtcatatggctaaacctggcaccaa

SEQ ID NO: 47 - DNA, 36 bp, Artificial Sequence; identical to SEQ ID NO: 20
  Synthesized in laboratory.  Not extracted from any organism.

SEQ ID NO: 48 - DNA, 15 bp, Artificial Sequence
  Synthesized in laboratory.  Not extracted from any organism.
aggacgctagcacac

SEQ ID NO: 49 - DNA, 36 bp, Artificial Sequence; identical to SEQ ID NO: 4
  Synthesized in laboratory.  Not extracted from any organism.

SEQ ID NO: 50 - DNA, 14 bp, Artificial Sequence
  Synthesized in laboratory.  Not extracted from any organism.
agcgatgctaccga

SEQ ID NO: 51 - DNA, 38 bp, Artificial Sequence; identical to SEQ ID NO: 1
  Synthesized in laboratory.  Not extracted from any organism.

SEQ ID NO: 52 - DNA, 25 bp, Artificial Sequence
  Synthesized in laboratory.  Not extracted from any organism.
aacgtgagtgctagcggagtcttaa

SEQ ID NO: 53 - DNA, 36 bp, Artificial Sequence; identical to SEQ ID NO: 4
  Synthesized in laboratory.  Not extracted from any organism.

SEQ ID NO: 54 - DNA, 47 bp, Artificial Sequence
  Synthesized in laboratory.  Not extracted from any organism.
aaaaaaaaaaaaaaaaaaaaaaaaaaaaaaaaagcgatgctaccgat

SEQ ID NO: 55 - DNA, 38 bp, Artificial Sequence; identical to SEQ ID NO: 1
  Synthesized in laboratory.  Not extracted from any organism.

SEQ ID NO: 56 - DNA, 25 bp, Artificial Sequence
  Synthesized in laboratory.  Not extracted from any organism.
aacgtgagtgctagcggagtcttaa

SEQ ID NO: 57 - DNA, 36 bp, Artificial Sequence; identical to SEQ ID NO: 4
  Synthesized in laboratory.  Not extracted from any organism.

SEQ ID NO: 58 - DNA, 36 bp, Artificial Sequence
  Synthesized in laboratory.  Not extracted from any organism.
aaaaaaaaaaaaaaaaaaaaaagcgatgctaccgat

(19) United States
(12) Patent Application Publication
(10) Pub. No.: US 2010/0105050 A1
(43) Pub. Date: Apr. 29, 2010

(54) REAL TIME DETECTION OF GENETIC SEQUENCES USING A BIPARTITE PROBE

(75) Inventors: Timothy A. Hodge, Eads, TN (US); Parasakthy Kumaravelu, Memphis, TN (US)

Correspondence Address: BUTLER, SNOW, O'MARA, STEVENS & CANNADA PLLC, 6075 POPLAR AVENUE, SUITE 500, MEMPHIS, TN, 38119, US

(73) Assignee: TRANSNETYX, INC., Cordova, TN (US)

(21) Appl. No.: 12/565,586

(22) Filed: Sep. 23, 2009

Related U.S. Application Data
(63) Continuation-in-part of application No. 10/233,804, filed on Sep. 3, 2002.
Application No. 10/233,804 is a continuation-in-part of application No. 09/945,952, filed on Sep. 4, 2001, now Pat. No. 7,011,943.
(60) Provisional application No. 60/230,701, filed on Sep. 7, 2000.

(51) Int. Cl. C12Q 1/68; C12P 19/34

(52) U.S. Cl. 435/6; 435/91.2

(57) ABSTRACT

The present invention concerns a method for detection or quantification of a Target Genetic Sequence in a Genetic Sample using a Bipartite Probe. A Bipartite Probe is made of a Target Binding Sequence capable of hybridizing a Target Genetic Sequence and a Nucleic Acid Binding Probe Sequence capable of being transcribed during PCR into the Capture Probe Sequence where it can hybridize with a Signal Generation Molecule. The Signal Generation Molecule participates in the PCR reaction and confers a fluorescent quality to the PCR amplified product. The reaction temperature of each PCR cycle is reduced below the Tm of the Quencher Oligonucleotide thereby allowing quenching of the fluorescence of the non-incorporated Signal Generation Molecule. Fluorescent detection of each PCR cycle establishes a quantitative PCR result.

### CROSS REFERENCE TO RELATED APPLICATIONS

This application is a CONTINUATION-IN-PART of U.S. application Ser. No. 10/233,804 filed Sep. 3, 2002; application Ser. No. 10/233,804 is a CONTINUATION-IN-PART of application Ser. No. 09/945,952 filed on Sep. 4, 2001 and issued as U.S. Pat. No. 7,011,943 on Mar. 14, 2006; said application Ser. No. 09/945,952 also claims priority under 35 U.S.C. §119(e), based on U.S. Provisional Application Ser. No. 60/230,371 filed Sep. 6, 2000. The entire disclosures of the above applications are hereby incorporated by reference.

### BACKGROUND OF THE INVENTION

1. Field of the Invention

This invention relates to a method of detection or quantification of genetic sequences in a Genetic Sample of nucleic acid using a Bipartite Probe and, more specifically, using the Bipartite Probe to produce Real Time Polymerase Chain Reaction (PCR) data. Real Time PCR is also known as quantitative PCR, or qPCR.

2. Description of Related Art

Different techniques may be employed in order to produce quantitative PCR data. Specifically, SYBR green nucleic acid stains (INVITROGEN, Carlsbad, Calif.), TAQMAN gene expression assays, BHQplus (Roche Molecular Systems, Pleasanton, Calif.), AMPLIFLUOR (Millipore Corp, Norcross, Ga.), Molecular Beacons (Public Health Research Institute, Newark N.J.), Scorpions Probes (DxS, Ltd. of Manchester, UK) and PLEXOR qPCR (Promega Corporation, Madison, Wis.) are also well-known sequence quantification detection technologies. Additionally, Kaspar (Kbiosciences, Hoddesdon, UK) technology is a well-known detection technology that produces end point data for biallelic single nucleotide polymorphisms (SNP).

The applications for this invention minor that of any quantitative sequence detection technology. However, this invention greatly reduces the cost and time to synthesize a new assay relative to other quantitative PCR technologies. Moreover, this invention is successful where other quantitative or detection technologies have been tried and failed.

SYBR green produces signal from the non-specific intercalation of a fluorescent stain preferential for double-stranded DNA. As PCR amplification occurs, the double stranded DNA product increases exponentially. The amplified DNA product incorporates the fluorescent stain. The fluorescence is quantified by instrumentation at each cycle of the PCR.

TAQMAN and BHQplus produce signal via PCR amplification and a specific dual-labeled probe. During the PCR amplification, the polymerase (i.e. Thermus aquaticus) exhibits 5′ exonuclease activity on a dual-labeled probe. The cleavage of the duel labeled probe separates the fluorescence molecule from the quencher, producing fluorescent signal.

The AMPLIFLUOR technology utilizes a single molecule that combines a primer, probe and quencher. The oligonucleotide contains the primer sequence at the 3′ end and a hairpin probe and quencher structure at the 5′ end. During the PCR amplification process the reporter and quencher are separated by transcription through the hairpin structure.

Molecular Beacons are dual-labeled Fluorescence Resonance Energy Transfer (FRET) probes incorporating a quencher and a fluorescent reporter molecule. The Molecular Probes have a short complementary sequence of bases at the 3′ and 5′ ends. These complementary sequences hybridize to form a stem structure which holds the reporter and quencher in close proximity. Molecular Beacons do not rely on probe destruction from 5′ exonuclease activity of Taq polymerase to generate its fluorescence.

Scorpion primers for PCR analysis combine primer and probe in one molecule, with the primer at the 3′ end and the probe contained within a hairpin-loop structure at the 5′ end. Scorpion primers do not require enzymatic cleavage of the probe during PCR cycling. Scorpions are probe/primer hybrids whose design, unlike other FRET probes, is such that they emit light only when bound to their complementary target sequence during PCR amplification.

PLEXOR primer technology for quantitative PCR is a technique that requires only two primers for sensitive and specific quantification of amplified DNA. PLEXOR primer technology utilizes the highly specific interaction between two modified nucleotides, which form a unique base pair when incorporated in double-stranded DNA and pair only with each other.

In PLEXOR reactions, one PCR primer is synthesized with an iso-dC residue and a fluorescent label at the 5′ end of the oligonucleotide. The second PCR oligonucleotide primer is unlabeled. Iso-dGTP nucleotides, modified to include a dabcyl quencher, are included in the PCR reaction mix. During the PCR amplification reaction only the dabcyl-iso-dGTP is incorporated at the position complementary to the iso-dC residue. The hybridization of the dabcyl-iso-dGTP in close proximity to the fluorescent label quenches the fluorescent signal.

The Kaspar technology produces biallelic end-point SNP data that is significantly different relative to quantitative PCR in the claimed invention. The PCR thermoprofile in combination with the specific oligonucleotides described in EP 1,726,664; and US 20070117108 will not produce quantitative PCR data. Further the assay mechanism between the claimed invention and the Kaspar technology are different. The Kaspar technology utilizes a competitive reaction between two oligonucleotides that have the same Target Binding Sequence except for the terminal nucleotide. The two oligonucleotides compete for hybridization of the same locus in the sample. The oligonucleotide with the terminal base mismatch will show less favorable hybridization. Conversely, the oligonucleotide that is perfectly matched to the genetic target will show preferential hybridization over the mismatch oligonucleotide. The Kaspar technology's two oligonucleotide discriminate which allele(s) are present in the sample via end-point data. In the current invention, there is not a competitive reaction between two oligonucleotides for the same locus, and quantitative PCR data is produced.

Quantitative PCR is more reliable than simple end-point data because it allows the detection of trace nucleic acid contamination that may be misinterpureted with simple end-point analysis, leading to an incorrect genotype.

All of the aforementioned technologies differ significantly in terms of mechanism and/or utility from the current invention.

### BRIEF SUMMARY OF THE INVENTION

The present invention is a process which detects or quantifies a Target Genetic Sequence in any Genetic Sample using a Bipartite Probe. A Bipartite Probe includes a Target Binding Sequence capable of hybridizing with the Target Genetic Sequence and an additional Nucleic Acid Binding Probe Sequence beyond the Target Binding Sequence. The Bipartite Probe hybridizes with the Target Genetic Sequence in the Genetic Sample, and the Nucleic Acid Binding Probe Sequence is transcribed in the PCR process, and the reverse compliment of the Bipartite Probe's Nucleic Acid Binding Probe Sequence (the Capture Probe Sequence) hybridizes to the Signal Generation Molecule. Unincorporated Signal Generation Molecules are rehybridized to the Quencher Oligonucleotide prior to fluorescent detection of each cycle of PCR.

We disclose a method for detection or quantification of a Target Genetic Sequence in a Genetic Sample by an assay which involves using a Bipartite Probe and at least one detectable Signal Generation Molecule. A Bipartite Probe has a Target Binding Sequence capable of hybridizing the Target Genetic Sequence in a sample of nucleic acid and a Nucleic Acid Binding Probe Sequence capable of being transcribed during PCR and the reverse compliment hybridizing with a Signal Generation Molecule. The transcribed reverse compliment is the Capture Probe Sequence. The Signal Generation Molecule is detectable by a variety of instruments.

Additionally, this invention provides a method to sequentially increase signal strength (i.e. quantitative PCR) in an assay for a Target Genetic Sequence in a Genetic Sample involving the steps of: hybridizing at least one detectable Signal Generation Molecule with a Bipartite Probe's Nucleic Acid Binding Probe Sequence reverse compliment (Capture Probe Sequence) in a PCR reactions solution, whereby the Signal Generation Molecule produces signal, as well as serving as a PCR primer for extension in subsequent cycles of PCR. Unincorporated Signal Generation Molecules are rehybridized to the Quencher Oligonucleotide prior to fluorescent detection of each cycle of PCR.

Similarly, this invention provides a method for the qPCR detection of more than one Target Genetic Sequence in a Genetic Sample involving the steps of: hybridizing more than one differentially detectable (FAM, VIC, TET, JOE, HEX, Cal 610, Cal 635, Tamra, Quazar 670 etc) Signal Generation Molecule with more than one Bipartites Probe's Nucleic Acid Binding Probe Sequences reverse compliments (Capture Probe Sequence) in a PCR reaction solution, whereby the Signal Generation Molecules produce detectably different fluorescence, as well as serving as a PCR primer for extension in subsequent cycles of PCR.

### BRIEF DESCRIPTION OF THE DRAWINGS

A more complete understanding of the invention and its advantages will be apparent from the following Description of the Preferred Embodiment(s) taken in conjunction with the accompanying drawings, wherein:

FIG. 1 depicts the bipartite probe.

FIG. 2 illustrates the activity of the bipartite probe bound to a genetic sample.

FIG. 3 illustrates the steps of the claimed methods.

FIG. 4 further illustrates the steps of the claimed method.

FIG. 5 shows the graphical results obtained in the course of performance of Example 1.

FIG. 6 shows the graphics results obtained for the Mutant Allele in the course of performance of Example 2.

FIG. 7 shows the graphics results obtained for the Housekeeping Allele in the course of performance of Example 2.

FIG. 8 shows the graphics results obtained for the Mutant Allele and Housekeeping Allele shown together in the course of performance of Example 2.

FIG. 9 shows the graphics results obtained in the course of performance of Example 3.

FIG. 10 shows the graphics results obtained Bacterial Neomycin in the course of performance of Example 4.

FIG. 11 shows the graphics results obtained for SNP Homozygous in the course of performance of Example 5.

FIG. 12 shows the graphics results obtained for SNP Herterozygous in the course of performance of Example 5.

FIG. 13 shows the graphics results obtained in the course of performance of Example 6.

FIG. 14 shows the graphics results obtained in the course of performance of Example 7.

FIG. 15 shows a stem loop structure for the bipartite as described.

FIG. 16 depicts the homodimer as described.

FIG. 17 depicts the stem loop structure as described for the nucleic acid binding probe sequence.

FIG. 18 depicts the stem loop structure for target binding sequence

FIG. 19 depicts the stem loop structure for the signal generating molecule.

FIG. 20 depicts the homodimer for the signal generating molecule.

FIG. 21 depicts the stem loop structure for signal generating molecule that hybridizes to the capture probe sequence.

FIG. 22 depicts the stem loop structure for the quencher oligonucleotide.

FIG. 23 depicts the homodimer for the quencher oligonucleotide.

FIG. 24 depicts the stem loop structure for the reverse primer.

FIG. 25 shows the homodimer for the reverse primer.

### DETAILED DESCRIPTION OF THE INVENTION

The present invention provides a method for discriminating a genetic sequence using a Bipartite Probe 1 to localize a Signal Generation Molecule 6. All patents, patent applications and articles discussed or referred to in this specification are hereby incorporated by reference. Incorporate U.S. Pat. No. 5,538,848; PCT WO00/41549; EP 0,909,823; PCT WO02/30946; PCT WO 00/49293; DE 10230948; EP 1,726,664; and US 20070117108.

The following terms and acronyms are used throughout the detailed description:

complementary—chemical affinity between nitrogenous bases as a result of hydrogen bonding. Responsible for the base pairing between nucleic acid strands. Klug, W. S. and Cummings, M. R. (1997) Concepts of Genetics, 5th ed., Prentice-Hall, Upper Saddle River, N.J. (hereby incorporated by reference)

DNA (deoxyribonucleic acid)—The molecule that encodes genetic information. DNA is a double-stranded molecule held together by weak bonds between base pairs of nucleotides. The four nucleotides in DNA contain the bases: adenine (A), guanine (G) cytosine (C), and thymine (T). In nature, base pairs form only between A and T and between G and C; thus the base sequence of each single strand can be deduced from that of its partner. DNA can be denatured and exist as a single stranded species.

genome—all the genetic material in a particular organism; its size is generally given as its total number of base pairs.

genomic DNA—all of the genetic information encoded in a cell. Lehninger, A. L., Nelson, D. L. Cox, M. M. (1993) Principles of Biochemistry, 2nd ed., Worth Publishers, New York, N.Y. Further this is meant to also include DNA, oligonucleotides, PCR amplicons, total RNA, mRNA, rRNA, trRNA, tRNA, mitochondrial DNA, cDNA, plasmid DNA, cosmid DNA and chloroplastic DNA from any prokaryote or eukaryote.

genotype—genetic constitution of an individual cell or organism.

recombinant DNA—A combination of DNA molecules of different origin that are joined using recombinant DNA technologies.

Target Genetic Sequence—includes a transgenic insert, a selectable marker, recombinant site or any gene or genetic segment from a prokaryote, eukaryote or viruses. Target Genetic Sequences can be either DNA, oligonucleotides, PCR amplicons, total RNA, mRNA, rRNA, trRNA, tRNA, mitochondrial DNA, cDNA, plasmid DNA, cosmid DNA and chloroplastic DNA.

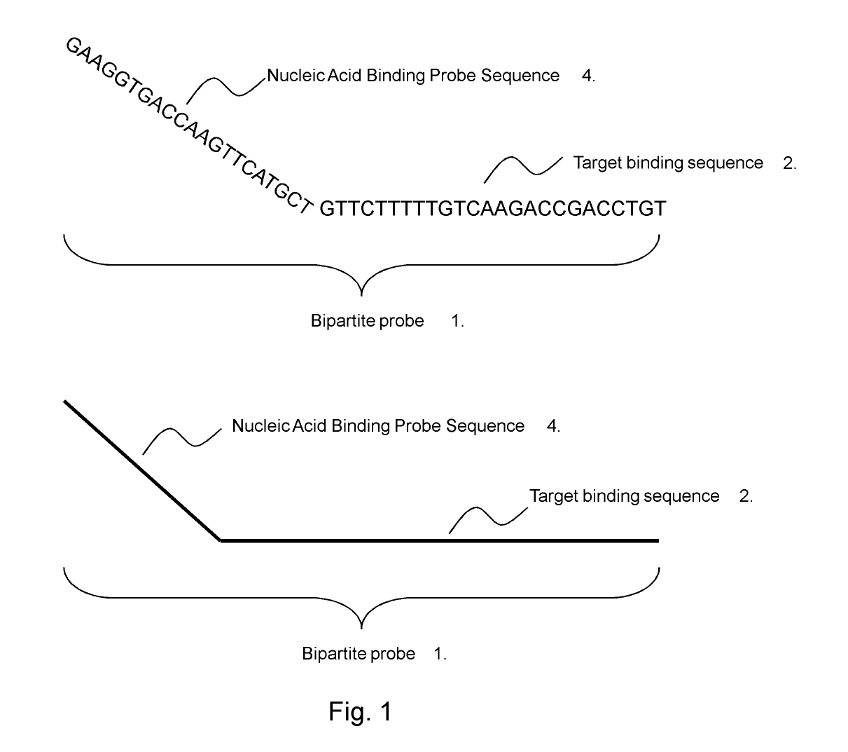

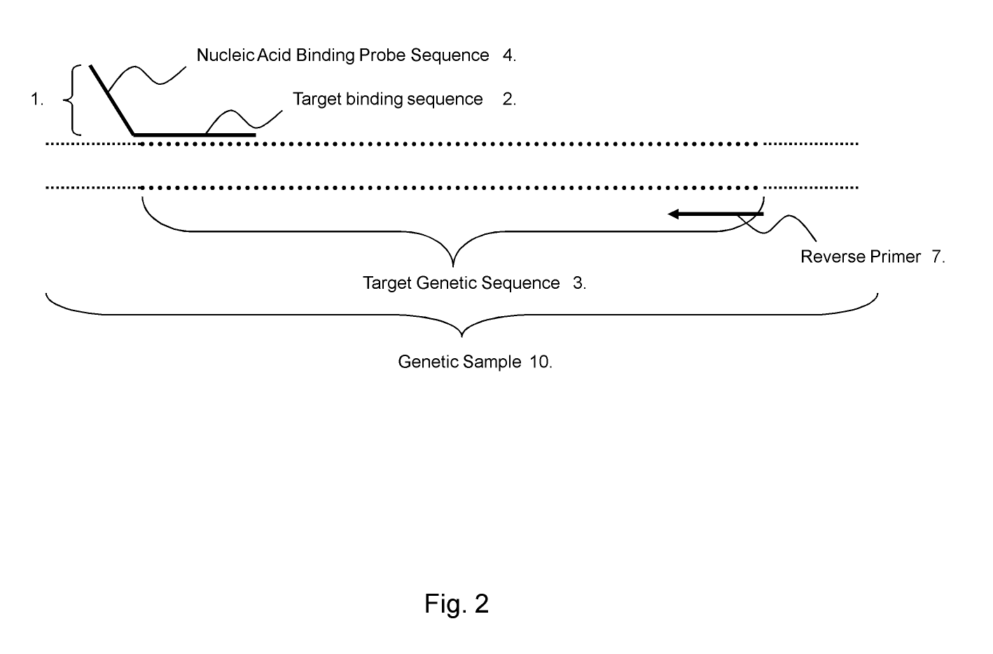

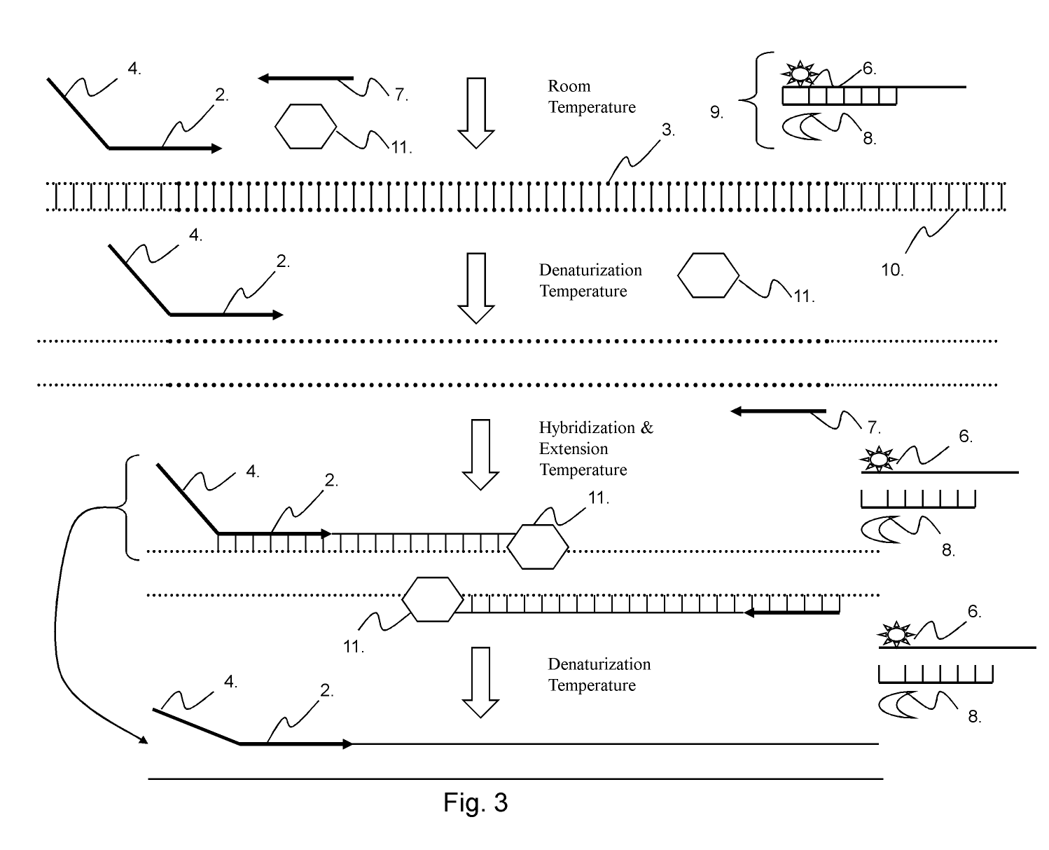

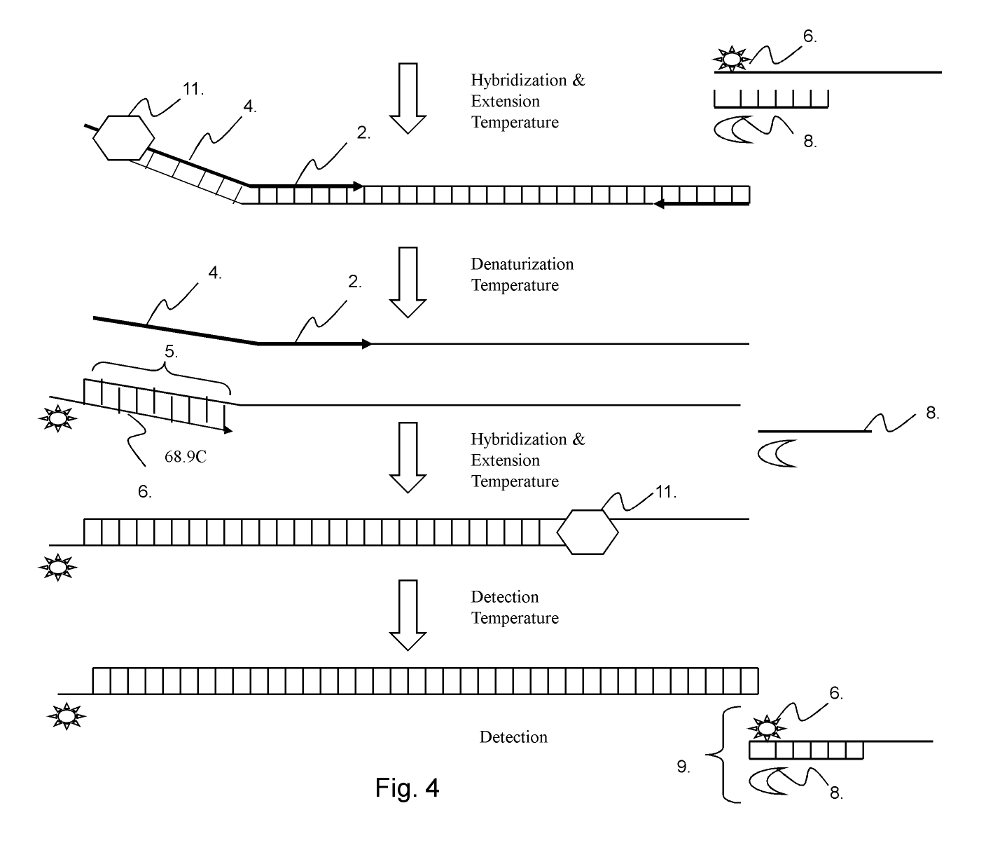

With reference to FIGS. 1, 2, 3 and 4 of the present invention, a process and method which detects and/or quantifies a Target Genetic Sequence 3 in any Genetic Sample 10 is depicted. The Genetic Sample 10 can include eukaryotic or prokaryotic genomic DNA, PCR amplicons, total RNA, mRNA, mitochondrial DNA, cDNA, plamid DNA, cosmid DNA and chloroplastic DNA. A Bipartite Probe 1 includes a Target Binding Sequence 2 capable of hybridizing with the Target Genetic Sequence 3 and an additional Nucleic Acid Binding Probe Sequence 4 adjacent to the Target Binding Sequence 2.

A Bipartite Probe 1 can function in a PCR reaction by having its Target Binding Sequence 2 function as either as a 3′ or 5′ primer. Further, the bipartites Nucleic Acid Binding Probe Sequence 4 confers a detectable and quantitative attribute via the transcribed Capture Probe Sequence 5. A Bipartite Probe 1 and an addition PCR primer 7 is hybridized, under PCR conditions, with the Genetic Sample 10 including the Target Genetic Sequence 3 and a detectable Signal Generation Molecule 6. The Signal Generation Molecule 6 oligonucleotide exists in a FRET Cassette 9, in the PCR mix, such that the fluorescent signal is substantially quenched by the Quencher Oligonucleotide 8 absent PCR amplification at the fluorescence detection temperature. The FRET Cassette is composed of a fluorescently labeled oligonucleotide, (i.e. Signal Generation Molecule 6) that is hybridized to a Quencher Oligonucleotide 8 at detection temperature. In the preferred embodiment, Biosearch Technologies (Novato, Calif.) BLACK HOLE QUENCHER oligonucleotides are utilized. During PCR, the polymerase 11 transcribes the denatured DNA, including the Nucleic Acid Probe Binding Sequence 4 of the Bipartite Probe 1. Moreover, the FRET Cassette 9 is denatured, and the fluorescently labeled Signal Generation Molecule 6 participates in the PCR reaction as a PCR primer. The Signal Generation Molecule 6 hybridizes to the reverse complement of the Bipartite Probe's 1 Nucleic Acid Binding Probe Sequence 4. The reverse compliment sequence created via PCR transcription of the bipartites Nucleic Acid Binding Probe Sequence 4 is the Capture Probe Sequence 5. The detectable Signal Generation Molecule 6 hybridizes with the Capture Probe Sequence 5.

A Capture Probe Sequence 5 is preferably about 20 bases in length. Longer or shorter Capture Probe Sequence 5 are possible. The length and sequence of the Target Binding Sequence 2, the additional Reverse Primer 7, the Capture Probe Sequence 5 vis-a-vis the Nucleic Acid Binding Probe Sequence 4 and the Signal Generation Molecule 6 will define the specificity of the assay for a particular size genome.

A plurality of Bipartite Probes 1 and Reverse Primers 7 and FRET Cassettes 9 can be combined together in the same assay for the detection of two or more Target Genetic Sequences 3. The practice of combining different Bipartites Probes 1, Reverse Primers 7 and FRET Cassettes 9 together for the detection of more than one Target Genetic Sequences 3 in a single assay or reaction is known as multiplexing. When Bipartite Probes 1 are multiplexed together, they typically have different Target Binding Sequences 2 and Reverse Primers 7 to confer specificity to unique Target Genetic Sequence 3. When Bipartite Probes 1 are multiplexed, they typically have different Nucleic Acid Binding Probe Sequences 4 to confer detection by a Signal Generation Molecule 6 labeled with different specific fluorescent labels. For example one FRET Cassette may have a quenched FAM label whereas another FRET Cassette may have a quenched Cal 560 label.

Alternatively, Bipartites 1 with different Target Binding Sequences 2 can have the same Nucleic Acid Binding Probe Sequences 4; however, in the event of positive amplification, it would be unknown which of the Target Genetic Sequences 3 amplified.

Specificity of the detection reaction is conferred by the base composition/sequence and length of the oligonucleotides reactants. The number of bases that compose the Target Binding Sequences 2 of the Bipartite Probe 1, the Reverse Primer 7 and the Signal Generation Molecule 6 can vary based on the size of the genome under study. The first consideration is to make sure the reactants oligonucleotide sequence is specific in terms of sequence and length for the Target Genetic Sequence 3 in the genome under study. For example, DNA has four bases (adenine, thymine, guanine and cytosine) which, when raised to the number of bases in the oligonucleotide sequence (eg. 4̂number of bases), should optimally be a number larger than the number of bases in the genome being studied. For instance, the number of base pairs in the mouse genome is approximately 3.0×10̂9. For consideration of a Bipartite Probe's 1 Target Binding Sequence 2 being 16 bases in length, the calculation would be 4̂16 which is 4.29×10̂9. Therefore, 16 bases should be adequate for this one oligonucleotide component of the reaction to be discriminatory for the mouse genome. Whereas, a target nucleic acid binding sequence of 10 bases would yield 4̂10 which is 1.05×10̂6. This is less than 3.0×10̂9, rendering 10 bases inadequate to be discriminatory for the mouse genome.

The optimal reaction temperature for the PCR reaction is dependent on the length and nucleotide composition of the different oligonucleotide elements of the Bipartite Probe 1, the Reverse Primer 7 and the FRET Cassette 9, specifically, the Signal Generation Molecule 6. For example, the successful quantitative detection of the reaction requires that the PCR reaction occur at a temperature that allows for the hybridization of the Bipartite Probes 1 Target Binding Sequence 2, the Reverse Primer 7 and the Signal Generation Molecule 6. This hybridization temperature, or sometimes called the annealing temperature, for each of the oligonucleotide species is typically less than their respective melting temperatures (Tm). The PCR reaction temperature should optimally be above the Tm of the Quencher Oligonucleotide 8 thereby allowing the Signal Generation Molecule 6 to participate in the PCR reaction and to impart a fluorescent quality to the PCR amplicon. However, the detection of the signal quantification optimally occurs at a temperature close to the Tm of the Quencher Oligonucleotide 8. It will work at temperatures as high as five degrees above the Tm, but temperatures below the Tm of the Quencher are preferred. This allows the Quencher Oligonucleotide 8 to hybridize with free Signal Generation Molecule 6, thereby quenching fluorescence from Signal Generation Molecule 6 that have not participated in the PCR reaction. This is the rehybridization, also called the re-naturization, of the Signal Generation Molecule 6 with the Quencher Oligonucleotide 8 which recreates the quenched FRET Cassette 9

Because of the hydrogen bonding between adenine and thymine and guanine cytosine, two and three bonds respectively, secondary structures can form. The secondary structures include stem loop structures, also known as homodimers. Stem loop structures form because of the affinity of one region of the oligonucleotide (i.e. Bipartite 1, Reverse Primer 7, Signal Generation Molecule 6 or Quencher Oligonucleotide 8) for another region of the same oligonucleotide. Homodimers may also show an unintended affinity for other oligonucleotides of the same species. Alternatively, heterodimers show an unintended affinity between different oligonucleotide species in a reaction.

In order to maximize the hybridization of the oligonucleotide species in the reaction, steps should be taken to eliminate or minimize secondary structures. Simply selecting a suitable alternative sequence, preferably one that is not rich in guanine and cytosine, often is all that is needed to produce a oligonucleotide species with no stable secondary structures. The application of heat sufficient to overcome the secondary structures is highly effective. However, the temperature to optimally hybridize the oligonucleotides and perform the PCR reaction should be higher than the temperature to reduce secondary structures. mFold or UNAfold are commercially available softwares that can predict secondary structures.

By way of example, the following shows the secondary characteristic of the flowing bipartite and its components. The bipartite:

5′-GAAGGTCGGAGTCAACGGATTCTCCCCAGTTCGCTCCA

has the following characteristics.

| SEQUENCE: (SEQ ID NO: 1) | | |
|---|---|---|
| 1 4 7 10 13 16 19 22 25 28 31 34 | | |
| 5′ GAA GGT CGG AGT CAA CGG ATT CTC CCC AGT TCG | | |
| 37 | | |
| CTC CA 3′ | | |
| 2.78 nM/OD | | |
| 32.62 ug/OD | | |
| MW = 11.7 k (one strand) | | |
| Primer to Target Tm (by % GC) = 87.5° C. | | |
| COMPOSITION: | | |
| A | 8.00 | 21.1% |
| C | 12.00 | 31.6% |
| G | 10.00 | 26.3% |
| T | 8.00 | 21.1% |
| X | .00 | 0.0% |
| A + T | 16.00 | 42.1% |
| C + G | 22.00 | 57.9% |

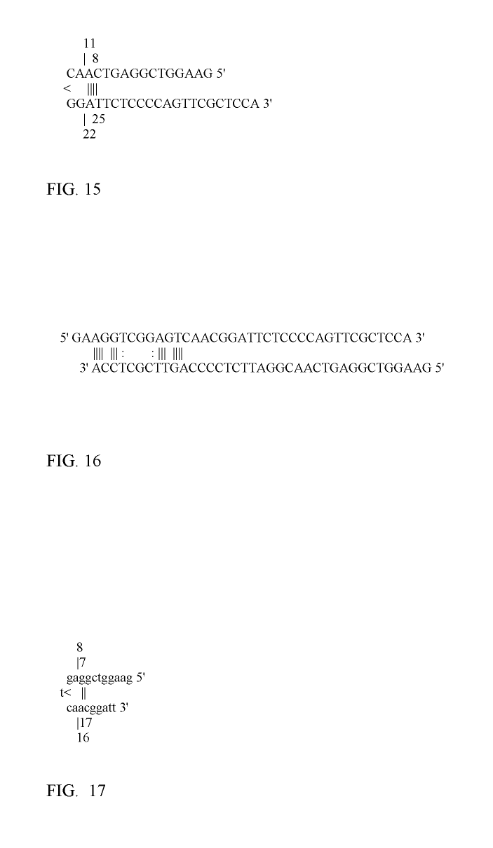

STEM LOOP STRUCTURE:
Shown in FIG. 15
G = −2.0 kcal/mol
loop Tm = 57° C.
HOMODIMER:
Shown in FIG. 16
Homodimer Tm = 29.6° C.

The Nucleic Acid Binding Probe Sequence of the bipartite which is:

| SEQUENCE: (SEQ ID NO: 2) | | |
|---|---|---|
| 1 4 7 10 13 16 19 | | |
| 5′ gaa ggt cgg agt caa cgg att 3′ | | |
| 4.66 nM/OD | | |
| 30.86 ug/OD | | |
| MW = 6.6 k (one strand) | | |
| Primer to Target Tm (by % GC) = 70.8° C. | | |
| COMPOSITION: | | |
| A | 6.00 | 28.6% |
| C | 3.00 | 14.3% |
| G | 8.00 | 38.1% |
| T | 4.00 | 19.0% |
| X | .00 | 0.0% |
| A + T | 10.00 | 47.6% |
| C + G | 11.00 | 52.4% |

STEM LOOP STRUCTURE:
Shown in FIG. 17
G = 0.5 kcal/mol
loop Tm = 7° C.
HOMODIMER:
No Homodimer

The Target Binding Sequence of the bipartite which is:

| SEQUENCE: (SEQ ID NO: 3) | | |
|---|---|---|
| 1 4 7 10 13 16 | | |
| 5′ CTC CCC AGT TCG CTC CA 3′ | | |
| 6.91 nM/OD | | |
| 35.38 ug/OD | | |
| MW = 5.1 k (one strand) | | |
| Primer to Target Tm (by % GC) = 68.3° C. | | |
| COMPOSITION: | | |
| A | 2.00 | 11.8% |
| C | 9.00 | 52.9% |
| G | 2.00 | 11.8% |
| T | 4.00 | 23.5% |
| X | .00 | 0.0% |
| A + T | 6.00 | 35.3% |
| C + G | 11.00 | 64.7% |

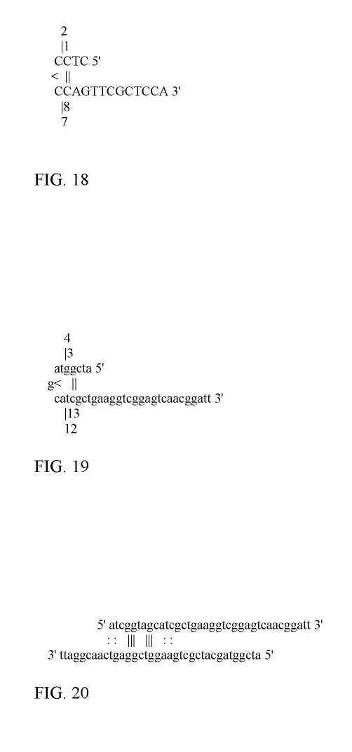

STEM LOOP STRUCTURE:
Shown in FIG. 18
G = 2.9 kcal/mol
No Stable Secondary Structure
HOMODIMER:
No Homodimer

The Signal Generating molecule:

| 5′-Cal560-atc ggt agc atc gct gaa ggt cgg agt | | |
|---|---|---|
| caa cgg att | | |
| SEQUENCE: (SEQ ID NO: 4) | | |
| 1 4 7 10 13 16 19 22 25 28 31 | | |
| 5′ atc ggt agc atc gct gaa ggt cgg agt caa cgg | | |
| 34 | | |
| att 3′ | | |
| 2.81 nM/OD | | |
| 31.56 ug/OD | | |
| MW = 11.2 k (one strand) | | |
| Primer to Target Tm (by % GC) = 84.4° C. | | |
| COMPOSITION: | | |
| A | 9.00 | 25.0% |
| C | 7.00 | 19.4% |
| G | 12.00 | 33.3% |
| T | 8.00 | 22.2% |
| X | .00 | 0.0% |
| A + T | 17.00 | 47.2% |
| C + G | 19.00 | 52.8% |

STEM LOOP STRUCTURE:
Shown in FIG. 19
G = 0.5 kcal/mol
loop Tm = 7° C.
HOMODIMER Shown in FIG. 20
Homodimer Tm = 12.2° C.

The portion of the Signal Generating molecule that hybridizes to the Capture Probe Sequence is the same sequence as the Nucleic Acid Binding Probe Sequence of the bipartite which is:

| SEQUENCE: (SEQ ID NO: 5) | | |
|---|---|---|
| 1 4 7 10 13 16 19 | | |
| 5′ gaa ggt cgg agt caa cgg att 3′ | | |
| 4.66 nM/OD | | |
| 30.86 ug/OD | | |
| MW = 6.6 k (one strand) | | |
| Primer to Target Tm (by % GC) = 70.8° C. | | |
| COMPOSITION: | | |
| A | 6.00 | 28.6% |
| C | 3.00 | 14.3% |
| G | 8.00 | 38.1% |
| T | 4.00 | 19.0% |
| X | .00 | 0.0% |
| A + T | 10.00 | 47.6% |
| C + G | 11.00 | 52.4% |

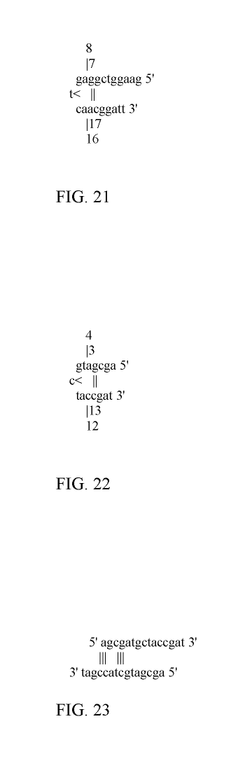

STEM LOOP STRUCTURE:
Shown in FIG. 21
G = 0.5 kcal/mol
loop Tm = 7° C.
HOMODIMER:
No Homodimer

The Quencher Oligonucleotide:

| 5′-agc gat gct acc gat-BHQ1 | | |
|---|---|---|
| SEQUENCE: (SEQ ID NO: 6) | | |
| 1 4 7 10 13 | | |
| 5′ agc gat gct acc gat 3′ | | |
| 6.83 nM/OD | | |
| 31.81 ug/OD | | |
| MW = 4.7 k (one strand) | | |
| Primer to Target Tm (by % GC) = 58.4° C. | | |
| COMPOSITION: | | |
| A | 4.00 | 26.7% |
| C | 4.00 | 26.7% |
| G | 4.00 | 26.7% |
| T | 3.00 | 20.0% |
| X | .00 | 0.0% |
| A + T | 7.00 | 46.7% |
| C + G | 8.00 | 53.3% |

STEM LOOP STRUCTURE Shown in FIG. 22:
G = 0.5 kcal/mol
loop Tm = 7° C.
HOMODIMER:
Shown in FIG. 23
Homodimer Not Stable

The Reverse Primer:

| AACGTGAGTGCTAGCGGAGTCTTAA | | |
|---|---|---|
| SEQUENCE: (SEQ ID NO: 7) | | |
| 1 4 7 10 13 16 19 22 25 | | |
| 5′ AAC GTG AGT GCT AGC GGA GTC TTA A 3′ | | |
| 3.97 nM/OD | | |
| 31.11 ug/OD | | |
| MW = 7.8 k (one strand) | | |
| Primer to Target Tm (by % GC) = 74.2° C. | | |
| COMPOSITION: | | |
| A | 7.00 | 28.0% |
| C | 4.00 | 16.0% |
| G | 8.00 | 32.0% |
| T | 6.00 | 24.0% |
| X | .00 | 0.0% |
| A + T | 13.00 | 52.0% |
| C + G | 12.00 | 48.0% |

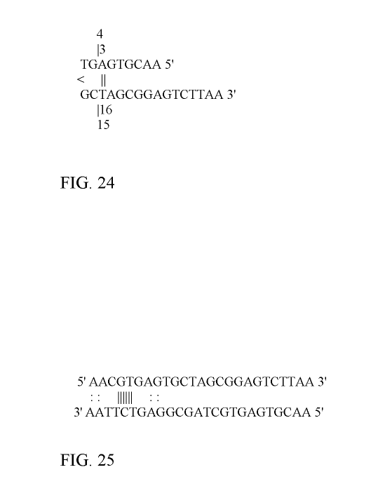

STEM LOOP STRUCTURE:
Shown in FIG. 24
G = 0.7 kcal/mol
loop Tm = 0° C.
HOMODIMER:
Shown in FIG. 25
Homodimer Tm = 14.2° C.

Examples of Bipartite Probes 1, complementary to single copy genes, are shown the table below:

TABLE 1

| | |
|---|---|
| Nr1h2-2 EX | 5′-GAAGGTGACCAAGTTCATGCTCGGAATTCCTCGAGTCTACTAGG |
| (SEQ ID NO: 8) | |
| Nr1h2-2 WT | 5′-GAAGGTGACCAAGTTCATGCTCGCACGCCCTAGGAAACC |
| (SEQ ID NO: 9) | |
| Gcgr WT | 5′-GAAGGTGACCAAGTTCATGCTAGCAGCTCCCACTCGAGCTTT |
| (SEQ ID NO: 10) | |
| Gcgr KO | 5′-GAAGGTGACCAAGTTCATGCTCAGCTCCCACTCGAGAAGTAC |
| (SEQ ID NO: 11) | |
| Cjun | 5′-GAAGGTCGGAGTCAACGGATTCTCCCCAGTTCGCTCCA |
| (SEQ ID NO: 12) | |
| Neo | 5′-GCGTCTTCTGTCCCATGCGTTCTTTTTGTCAAGACCGACCTGT |
| (SEQ ID NO: 13) | |

Examples of Reverse Primers.

TABLE 2

| | |
|---|---|
| Nr1h2-2 EX | 5′-GGCCAGGGCTGGGACACAAAA |
| (SEQ ID NO: 14) | |
| Nr1h2-2 WT | 5′-GGACAGAGCCCACCCAGGCTA |
| (SEQ ID NO: 15) | |
| Gcgr WT | 5′-CCTGTACCCAGATATGTCCTTCAGTA |
| (SEQ ID NO: 16 | |
| Gcgr KO | 5′-CACCTGACGCGAAGTTCCTATACTT |
| (SEQ ID NO: 17 | |
| Cjun | 5′-AACGTGAGTGCTAGCGGAGTCTTAA |
| (SEQ ID NO: 18) | |
| Neo | 5′-GCTGCCTCGTCCTGCAGTTCAT |
| (SEQ ID NO: 19) | |

TABLE 3

| | | |
|---|---|---|
| FRET Cassette 1 | Signal | 5′-FAM- |
| (SEQ ID NO: 20) | Generation | GTGTGCTAGCGTCCTGAAGGTGACCAAGTTCATGCT |
| | Molecule | |
| FRET Cassette 1 | Quencher | 5′-AGGACGCTAGCACAC-BHQ |
| (SEQ ID NO: 21) | Oligo- | |
| | nucleotide | |
| FRET Cassette 2 | Signal | 5′-Cal 560- |
| (SEQ ID NO: 22) | Generation | ATCGGTAGCATCGCTGAAGGTCGGAGTCAACGGATT |
| | Molecule | |
| FRET Cassette 2 | Quencher | 5′-AGCGATGCTACCGAT-BHQ |
| (SEQ ID NO: 23) | Oligo- | |
| | nucleotide | |
| FRET Cassette 3 | Signal | 5′-FAM- |
| (SEQ ID NO: 24) | Generation | GGCCGACTCACTGCGCGTCTTCTGTCCCATGC |
| | Molecule | |
| FRET Cassette 3 | Quencher | 5′-GCAGTGAGTCGGCC-BHQ |
| (SEQ ID NO: 25) | Oligo- | |
| | nucleotide | |

The PCR thermoprofile is designed in such a way to allow the denaturing of the Target Genetic Sequence 3, the hybridization of the Bipartite Probe 1 and Reverse Primer(s) 7, the extension of the polymerase 11 for transcribing Nucleic Acid Binding Probe Sequence 4, the denaturization of the FRET Cassette 9, the hybridization of the Signal Generation Molecule 6 to the Capture Probe Sequence 5, and the hybridization of the Quencher Oligonucleotide 8 to the non-incorporated Signal Generation Molecule 6 prior to the reading by the Real Time PCR instrumentation of each cycle.

In the preferred embodiment, 2.0 μl of PCR Master Mix is added to a well of a 384 PCR well plate. The MasterMix contains 0.1 μM Signal Generation Molecule 6, 0.5 μM Quencher Oligonucleotide 8, 1.0 μM of Reverse Primer 7 and 1.0 μM of Bipartite Probe 1 and 5 μM of Rhodamine passive reference dye, as well as traditional dNTPs, Polymerase, PCR buffer/salts. In the most preferred embodiment, the KASPar 2×PCR Master Mix (Cat# KASPar-5000) is purchased, which contains the FRET cassette, Rhodamine passive reference dye, salts and dNTPS. Additionally, 2.0 μl of 5-20 ng/μl concentration DNA from a Genetic Sample is added to a well of a 384 PCR well plate.

The DNA and FRET Cassette 9 is denatured in the Real Time PCR thermocycler via heating at 94° C. for 15 minutes. This heating process disrupts the hydrogen bonds between adenine, cytosine, guanine and thymine/uracil. The Bipartite Probe 1 and Reverse Primer 7 specifically hybridize to the Target Genetic Sequence 3 of the Genetic Sample 10 during the transition from 94° C. to 47° C. Additionally, the polymerase becomes active and extends the amplicon creating the Capture Probe Sequence 5. The Signal Generation Molecule 6 specifically hybridizes to the Capture Probe Sequence 5. The temperature decreases to below the Tm of the Quencher Oligonucleotide sequences, such as 47° C. The Quencher Oligonucleotide 8 in excess hybridizes and quenches the fluorescent signal of any Signal Generation Molecule 6 that did not get incorporated during PCR. When the Quencher Oligonucleotide 8 has hybridized to the unincorporated Signal Generation Molecule, the Real Time PCR instrumentation then measures the amount of fluorescence. The thermocycling continues between 94° C. for 20 seconds and 47° C. for 60 seconds 39 more times. The end result is a Real Time PCR plot of the Target Genetic Sequence.

### Example 1

### Bipartite Probe Quantitative Amplification of Single Copy Eukaryotic (Mouse) DNA Sequence

A Bipartite Probe oligonucleotide with a sequence of:

(SEQ ID NO: 26)

5′-GAAGGTGACCAAGTTCATGCTCGACATGACTCAGGATATGAAGTT,

a Reverse Primer with a sequence of:

| | |
|---|---|
| 5′-CCCACATCTTCTGCAAAGAACACCAA, | (SEQ ID NO: 27) |

a Signal Generation Molecule with a sequence of:

(SEQ ID NO: 28)

5′-FAM-GTGTGCTAGCGTCCTGAAGGTGACCAAGTTCATGCT

and a Quencher Oligonucleotide with a sequence of:

| | |
|---|---|
| 5′-AGGACGCTAGCACAC-BHQ | (SEQ ID NO: 29) |

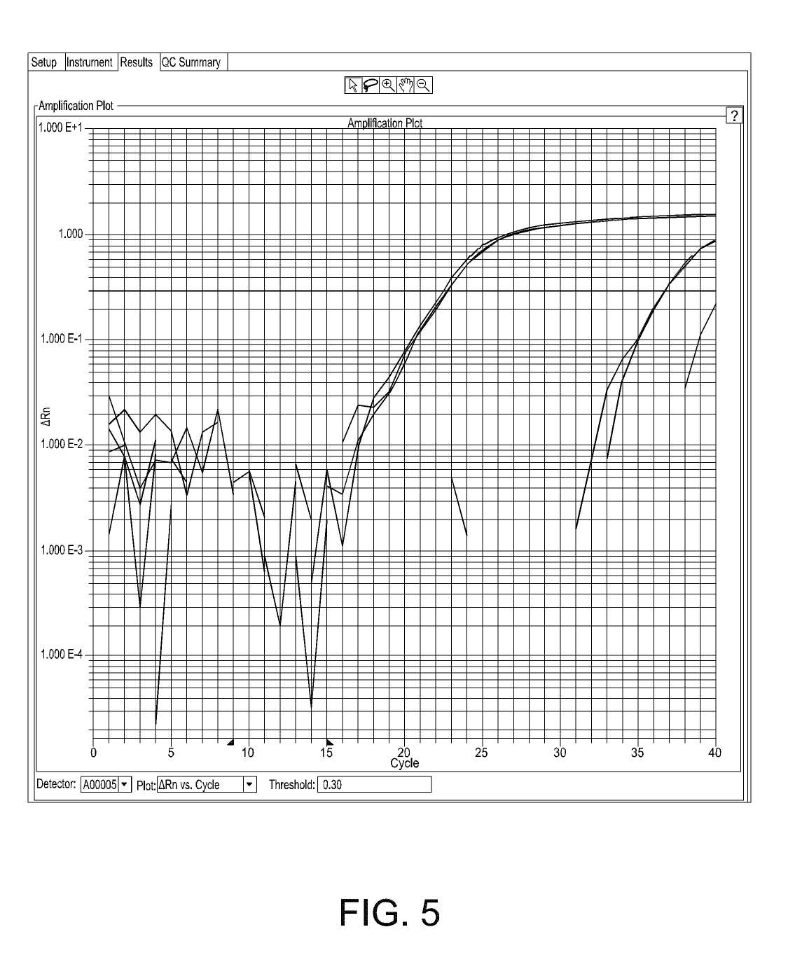

The oligonucleotides were ordered from an oligonucleotide vendor. All of the oligonucleotides were made to 100 μM stock concentrations. Working stocks were made by adding 12.0 μl of Bipartite Probe and 12 μl of Reverse Primer to 76 μl of PCR grade water. Further, 1.0 μl of Signal Generation Molecule and 5.0 μl or Quencher Oligonucleotide were added to 1000.0 μl of CLONTECH's Titanium Taq Master Mix (Cat# 639210). A reaction mix was made for 10 samples by combining 20.0 μl of 2× Titanium Taq Master Mix (which contained the FRET Cassette) with 0.55 μl of the working stock were added together. To each well of an APPLIED BIOSYSTEMS 384 PCR plate (Cat #4343814) was added 2.0 μl of 10 ng/μl mouse genomic DNA samples and 2 μl of the reactions mix. Three positive DNA samples, three negative DNA samples and two No Template Controls (which in this case was PCR grade water) were added to the reaction wells of the 384 wellplate. The APPLIED BIOSYSTEMS 7900 Real Time PCR thermocycler conditions were set such that there was a 15 denaturization step at 94° C. The denaturization step was followed by 40 cycles of 94° C. for 20 seconds and 47° C. for one minute. The APPLIED BIOSYSTEMS 7900 was run with 9600 emulation mode. The Real Time PCR plot shown in FIG. 5 was acquired. In this typical Real-Time PCR or Quantitative PCR data, each cycle of the PCR reaction is represented on the X-Axis, and the fluorescence is represented on the Y-Axis. The threshold is represented by the green bar intersecting the plots. The Ct data for each sample is collected where the fluorescent amplification plot for each sample intersects the threshold.

### Example 2

### Bipartite Probe Quantitative Amplification of Two Single copy Mouse DNA Sequence (Multiplex)

A endogenous/mutation allele Bipartite Probe oligonucleotide with a sequence of:

(SEQ ID NO: 30)

5′-GAAGGTGACCAAGTTCATGCTCGACATGACTCAGGATATGAAGTT,

a endogenous/mutation allele Reverse Primer with a sequence of:

| | |
|---|---|
| 5′-CCCACATCTTCTGCAAAGAACACCAA, | (SEQ ID NO: 31) |

a Signal Generation Molecule with a sequence of:

(SEQ ID NO: 32)

5′-FAM-GTGTGCTAGCGTCCTGAAGGTGACCAAGTTCATGCT

and a Quencher Oligonucleotide with a sequence of:

| | |
|---|---|
| 5′-AGGACGCTAGCACAC-BHQ | (SEQ ID NO: 33) |

A endogenous housekeeping allele bipartite oligonucleotide with a sequence of:

(SEQ ID NO: 34)

5′-GAAGGTCGGAGTCAACGGATTCTCCCCAGTTCGCTCCA,

a endogenous housekeeping allele Reverse Primer with a sequence of:

| | |
|---|---|
| 5′-AACGTGAGTGCTAGCGGAGTCTTAA, | (SEQ ID NO: 35) |

a Signal Generation Molecule with a sequence of:

(SEQ ID NO: 36)

5′-Cal 560 (or Vic fluorescent analog)-
ATCGGTAGCATCGCTGAAGGTCGGAGTCAACGGATT

and a Quencher Oligonucleotide with a sequence of:

(SEQ ID NO: 37)

5′-AGCGATGCTACCGA-BHQ (or quencher analog)

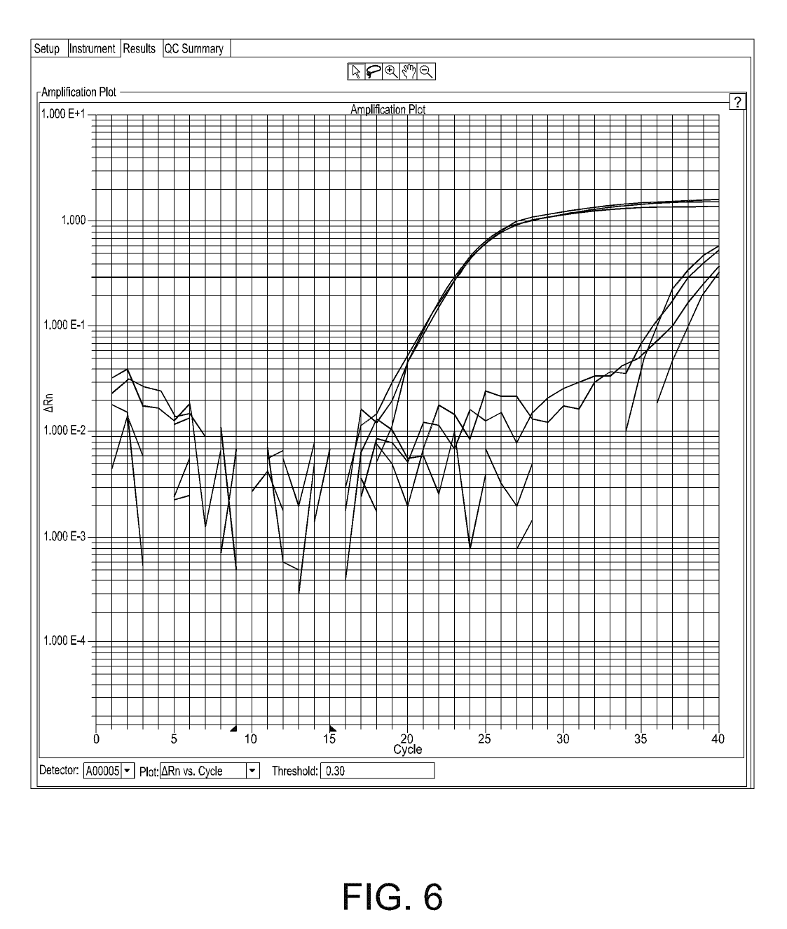

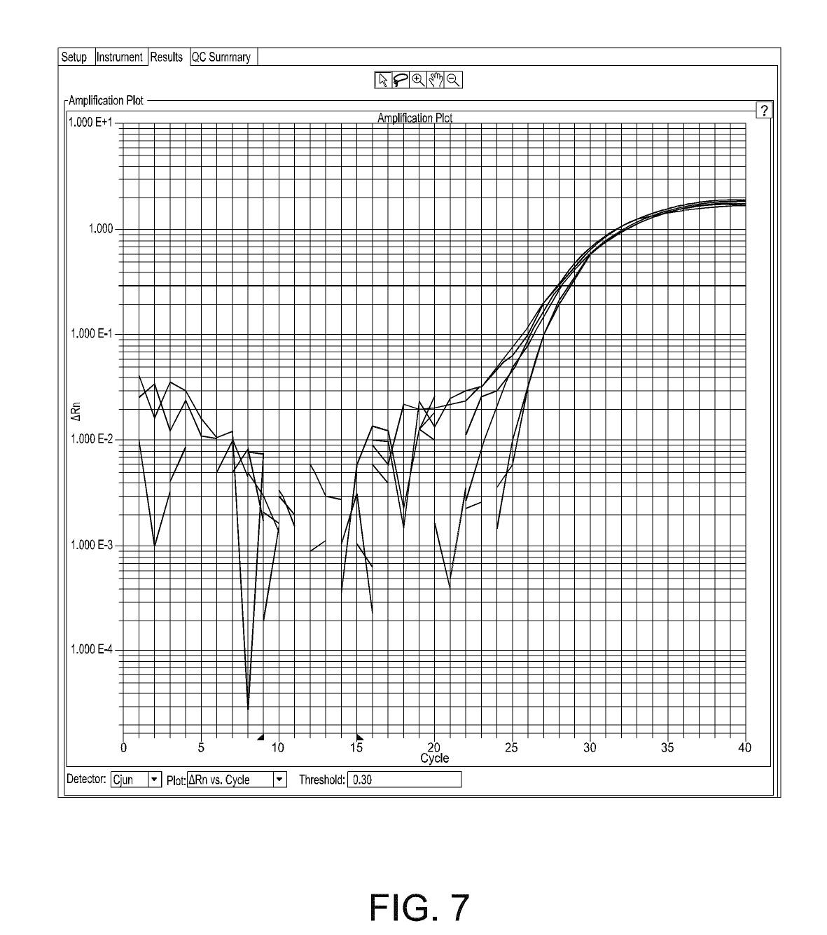

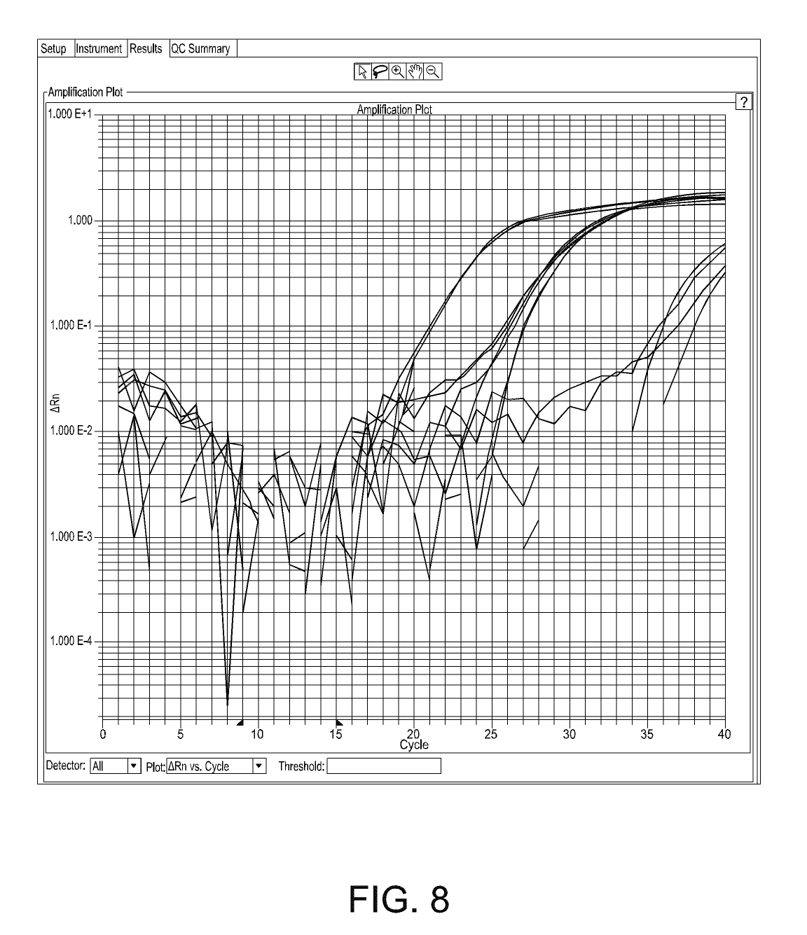

The oligonucleotides were ordered from an oligonucleotide vendor. All of the oligonucleotides were made to 100 μM stock concentrations. Working stocks were made by adding 12 μl of endogenous/mutation allele bipartite, 12 μl of endogenous housekeeping allele bipartite, 12 μl of endogenous/mutation allele Reverse Primer and 12 μl of endogenous housekeeping allele Reverse Primer to 52 μl of PCR grade water. 20 μl of KASPar 2× Master Mix, 0.55 μl of working stock and 0.32 μl of 50 mM magnesium chloride were added together to create the reaction mix. To each well of an APPLIED BIOSYSTEMS 384 PCR plate (Cat #4343814) was added 2 μl of 10 ng/μl mouse genomic DNA samples and 2 μl of the reactions mix. Three positive DNA samples, three negative DNA samples and two No Template Controls (which in this case was PCR grade water) were added to the reaction wells of the wellplate. The APPLIED BIOSYSTEMS 7900 Real Time PCR thermocycler conditions were set such that there was a 15 minute denaturization step at 94° C. The denaturization step was followed by 40 cycles of 94° C. for 20 seconds and 47° C. for one minute. The APPLIED BIOSYSTEMS 7900 was run with 9600 emulation mode. The Real Time PCR plots shown in FIG. 6 (mutant alleles), FIG. 7 (housekeeping alleles), and FIG. 8 (mutant allele and housekeeping allele shown together) were acquired.

FIG. 6 illustrates typical Real-Time PCR or Quantitative PCR data for mutant alleles. Each cycle of the PCR reaction is represented on the X-Axis, and the fluorescence is represented on the Y-Axis. The threshold is represented by the green bar intersecting the plots. The Ct data for each sample is collected where the fluorescent amplification plot for each sample intersects the threshold.

FIG. 7 illustrates typical Real-Time PCR or Quantitative PCR data for the Housekeeping Allele. Each cycle of the PCR reaction is represented on the X-Axis, and the fluorescence is represented on the Y-Axis. The threshold is represented by the green bar intersecting the plots. The Ct data for each sample is collected where the fluorescent amplification plot for each sample intersects the threshold.

FIG. 8 illustrates typical Real-Time PCR or Quantitative PCR data with mutant allele and housekeeping allele shown together. Each cycle of the PCR reaction is represented on the X-Axis, and the fluorescence is represented on the Y-Axis. The Ct data for each sample is collected where the fluorescent amplification plot for each sample intersects the threshold.

### Example 3

### Bipartite Probe Quantitative Amplification of Single Copy Eukaryotic (Human) DNA Sequence

A Bipartite Probe oligonucleotide with a sequence of:

(SEQ ID NO: 38)

5′-GAAGGTGACCAAGTTCATGCTGTCCACCTTCCAGCAGATGTG

a Reverse Primer with a sequence of:

| | |
|---|---|
| 5′-GGAGGGGCCGGACTCGTCAT | (SEQ ID NO: 39) |

a Signal Generation Molecule with a sequence of:

(SEQ ID NO: 40)

5′-FAM-GTGTGCTAGCGTCCTGAAGGTGACCAAGTTCATGCT

and a Quencher Oligonucleotide with a sequence of:

| | |
|---|---|
| 5′-AGGACGCTAGCACAC-BHQ | (SEQ ID NO: 41) |

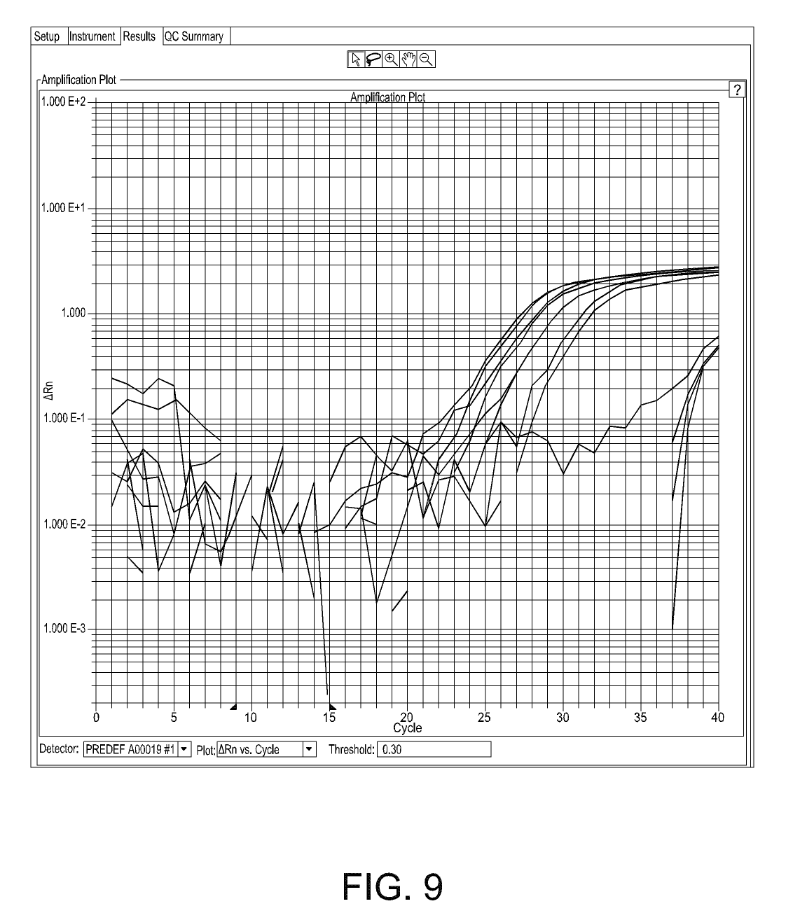

The oligonucleotides were ordered from an oligonucleotide vendor. All of the oligonucleotides were made to 100 μM stock concentrations. Working stocks were made by adding 12 μl of Bipartite Probe and 12 μl of Reverse Primer to 76 μl of PCR grade water. 40 μl of KASPar 2× Master Mix, 1.10 μl of working stock and 0.64 μl of 50 mM magnesium chloride were added together to create the reaction mix. To each well of an APPLIED BIOSYSTEMS 384 PCR plate was added 2 μl of 10 ng/μl human genomic DNA samples and 2 μl of the reactions mix. Eight positive DNA samples, and two No Template Controls (which in this case was PCR grade water) were added to the reaction wells of the wellplate. The APPLIED BIOSYSTEMS 7900 Real Time PCR thermocycler conditions were set such that there was a 15 minute denaturization step at 94° C. The denaturization step was followed by 40 cycles of 94° C. for 20 seconds and 47° C. for one minute. The APPLIED BIOSYSTEMS 7900 was run with 9600 emulation mode. The Real Time PCR plot shown in FIG. 9 was acquired.

FIG. 9 illustrates typical Real-Time PCR or Quantitative PCR data. Each cycle of the PCR reaction is represented on the X-Axis, and the fluorescence is represented on the Y-Axis. The threshold is represented by the green bar intersecting the plots. The Ct data for each sample is collected where the fluorescent amplification plot for each sample intersects the threshold.

### Example 4

### Bipartite Probe Quantitative Amplification of Bacterial (Prokaryote) DNA Sequence

A Bipartite Probe oligonucleotide with a sequence of:

(SEQ ID NO: 42)

5′-GAAGGTGACCAAGTTCATGCTGTTCTTTTTGTCAAGACCGACCTGT,

a Reverse Primer with a sequence of:

| | |
|---|---|
| 5′-GCTGCCTCGTCCTGCAGTTCAT, | (SEQ ID NO: 43) |

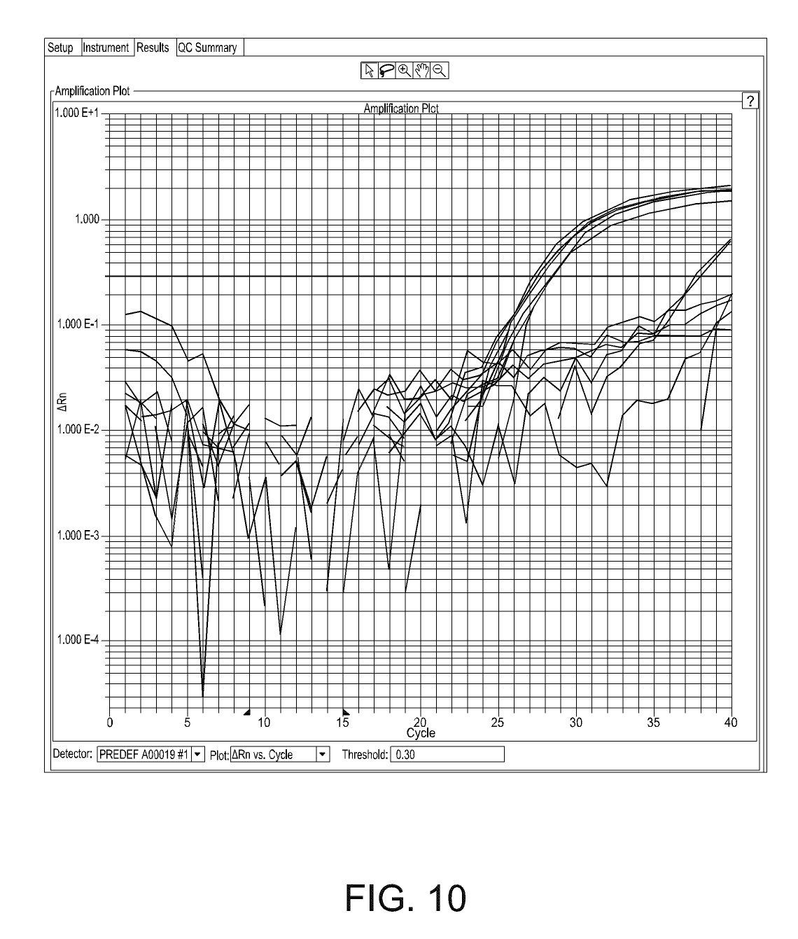

The oligonucleotides were ordered from an oligonucleotide vendor. All of the oligonucleotides were made to 100 μM stock concentrations. Working stocks were made by adding 12 μl of Bipartite Probe and 12 μl of Reverse Primer to 76 μl of PCR grade water. 40 μl of KASPar 2× Master Mix, 1.10 μl of working stock and 0.64 μl of 50 mM magnesium chloride were added together to create the reaction mix. To each well of an APPLIED BIOSYSTEMS 384 PCR plate was added 2 μl of 10 ng/μl DNA containing the neomycin bacterial sequence and 2 μl of the reactions mix. Eight positive DNA samples, eight negative DNA samples and two No Template Controls (which in this case was PCR grade water) were added to the reaction wells of the wellplate. The APPLIED BIOSYSTEMS 7900 Real Time PCR thermocycler conditions were set such that there was a 15 minute denaturization step at 94° C. The denaturization step was followed by 40 cycles of 94° C. for 20 seconds and 37° C. for one minute. The APPLIED BIOSYSTEMS 7900 was run with 9600 emulation mode. The Real Time PCR plot in FIG. 10 was acquired.

FIG. 10 illustrates typical Real-Time PCR or Quantitative PCR data for Bacterial Neomycin. Each cycle of the PCR reaction is represented on the X-Axis, and the fluorescence is represented on the Y-Axis. The threshold is represented by the green bar intersecting the plots. The Ct data for each sample is collected where the fluorescent amplification plot for each sample intersects the threshold.

### Example 5

### Bipartite Probes and SNP Real Time Amplification of Human DNA

A quantitative SNP assay was developed by designing two Bipartite Probe oligonucleotides with a sequence differing by only one nucleotide. The Bipartite Probe sequences were:

(SEQ ID NO: 44)

5′-GAAGGTGACCAAGTTCATGCTCACTTTGGTGGGTAAAAGAAGGC
and

(SEQ ID NO: 45)

5′-GAAGGTCGGAGTCAACGGATTCCACTTTGGTGGGTAAAAGAAGGT,

a Reverse Primer with a sequence of:

| | |
|---|---|
| 5′-GTCATATGGCTAAACCTGGCACCAA, | (SEQ ID NO: 46) |

a Signal Generation Molecule for allele 1 had a sequence of:

(SEQ ID NO: 47)
5′-FAM (or fluorescent analog)-
GTGTGCTAGCGTCCTGAAGGTGACCAAGTTCATGCT

and a Quencher Oligonucleotide for allele 1 had a sequence of:

(SEQ ID NO: 48)
5′-AGGACGCTAGCACAC-BHQ (or quencher analog)

a Signal Generation Molecule for allele 2 had a sequence of:

(SEQ ID NO: 49)
5′-Cal 560 (or Vic fluorescent analog)-
ATCGGTAGCATCGCTGAAGGTCGGAGTCAACGGATT

and a Quencher Oligonucleotide for allele 2 had a sequence of:

(SEQ ID NO: 50)
5′-AGCGATGCTACCGA-BHQ (or quencher analog)

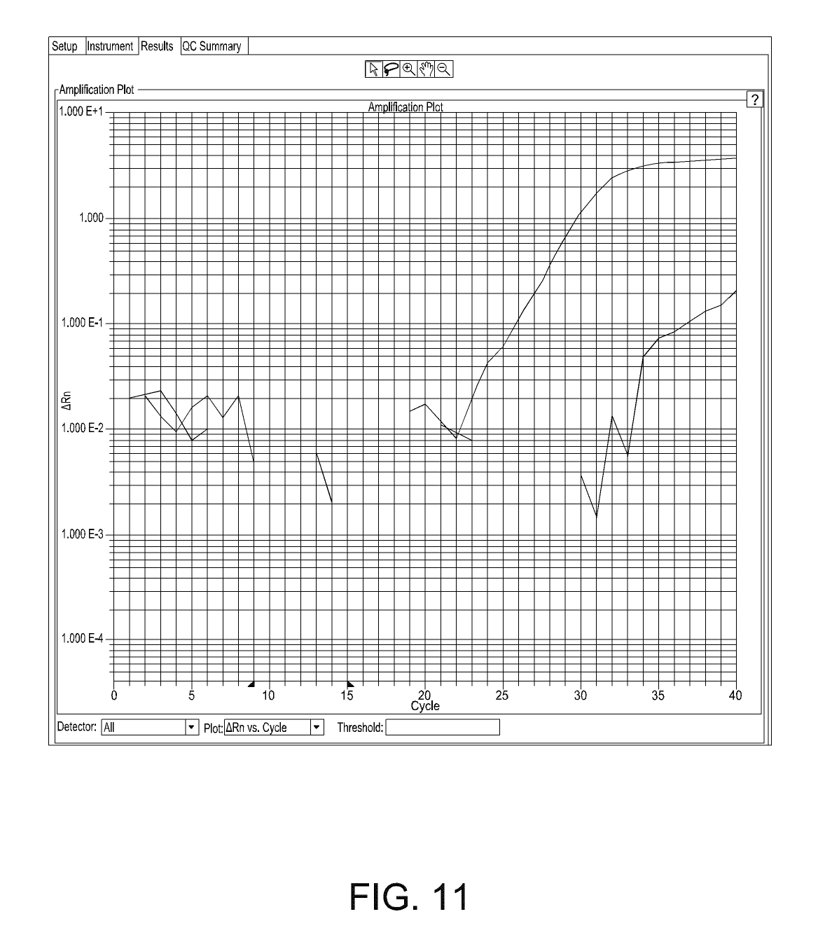

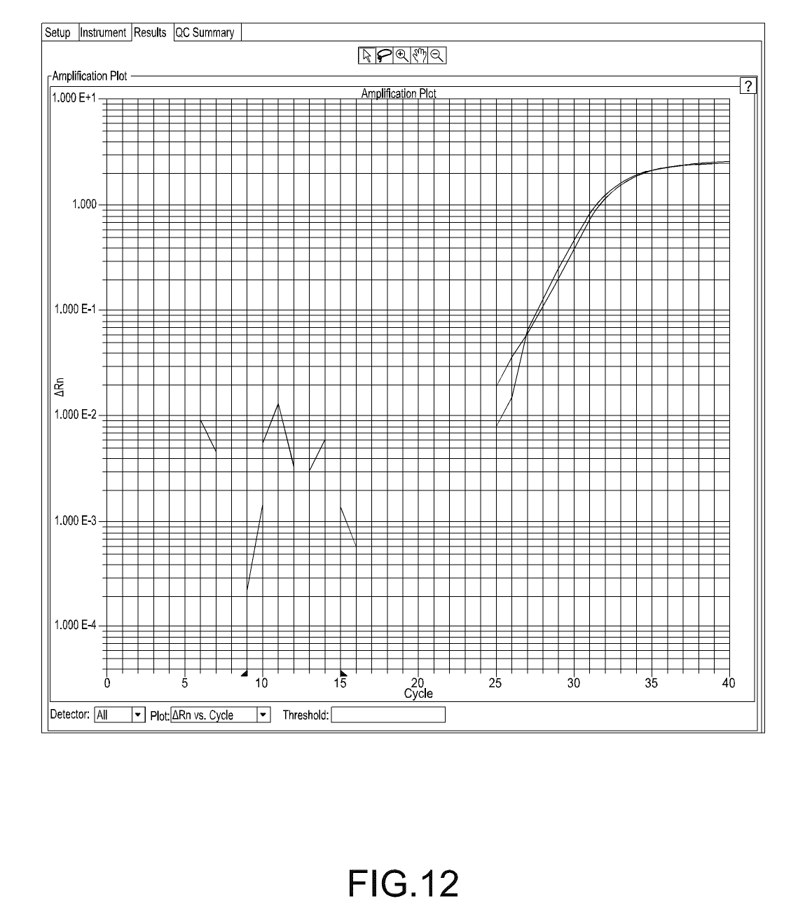

The oligonucleotides were ordered from an oligonucleotide vendor. All of the oligonucleotides were made to 100 μM stock concentrations. Working stocks were made by adding 12 μl of Allele 1 Bipartite Probe, 12 μl of Allele 2 Bipartite Probe and 24 μl of Reverse Primer to 52 μl of PCR grade water. 20 μl of KASPar 2× Master Mix, 0.55 μl of working stock and 0.32 μl of 50 mM magnesium chloride were added together to create the reaction mix. To each well of an APPLIED BIOSYSTEMS 384 PCR plate was added 2 μl of 10 ng/μl human genomic DNA samples and 2 μl of the reactions mix. One heterzygous DNA sample, one homozygous DNA samples and two No Template Controls (which in this case was PCR grade water) were added to the reaction wells of the wellplate. The APPLIED BIOSYSTEMS 7900 Real Time PCR thermocycler conditions were set such that there was a 15 minute denaturization step at 94° C. The denaturization step was followed by 40 cycles of 94° C. for 20 seconds and 37° C. for one minute. The APPLIED BIOSYSTEMS 7900 was run with 9600 emulation mode. The Real Time PCR plots shown in FIG. 11 and FIG. 12 were acquired.

FIG. 11 illustrates typical Real-Time PCR or Quantitative PCR data for SNP Homozygous. Each cycle of the PCR reaction is represented on the X-Axis, and the fluorescence is represented on the Y-Axis. The Ct data for each sample is collected where the fluorescent amplification plot for each sample intersects the threshold.

FIG. 12 illustrates typical Real-Time PCR or Quantitative PCR data for SNP Heterozygous. Each cycle of the PCR reaction is represented on the X-Axis, and the fluorescence is represented on the Y-Axis. The Ct data for each sample is collected where the fluorescent amplification plot for each sample intersects the threshold.

### Example 6

### Bipartite Probe Quantitative Amplification of Single Copy Eukaryotic (Mouse) DNA Sequence with FRET Oligonucleotides with Equal Melting Temperatures

A Bipartite Probe oligonucleotide with a sequence of:

(SEQ ID NO: 51)
5′-GAAGGTCGGAGTCAACGGATTCTCCCCAGTTCGCTCCA,

a Reverse Primer with a sequence of:

| | |
|---|---|
| 5′-AACGTGAGTGCTAGCGGAGTCTTAA, | (SEQ ID NO: 52) |

a Signal Generation Molecule with a sequence of:

(SEQ ID NO: 53)

5′-Gold 540-atc ggt agc atc gct gaa ggt cgg agt
caa cgg att

5′- and a Quencher Oligonucleotide with a sequence of:

(SEQ ID NO: 54)

5′ 5′-aa aaa aaa aaa aaa aaa aaa aaa aaa aaa aaa
agc gat gct acc gat-BHQ

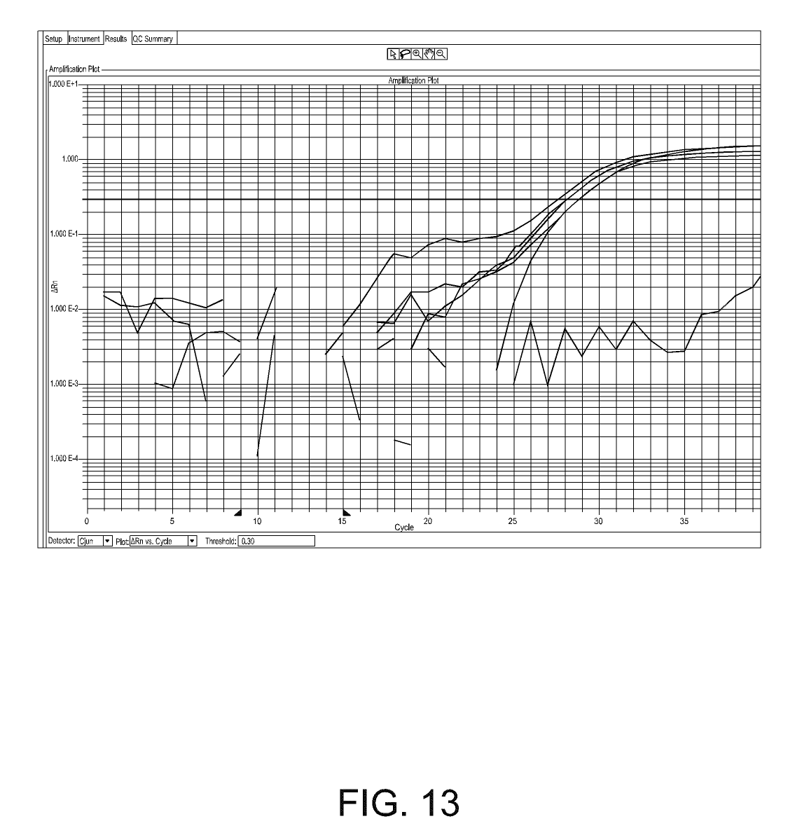

The oligonucleotides were ordered from an oligonucleotide vendor. All of the oligonucleotides were made to 100 μM stock concentrations. Working stocks were made by adding 12.0 μl of Bipartite Probe and 12 μl of Reverse Primer to 76 μl of PCR grade water. Further 1.0 μl of Signal Generation Molecule and 5.0 μl or Quencher Oligonucleotide were added to 1000.0 μl of CLONTECH's Titanium Taq Master Mix (Cat# 639210). A reaction mix was made for 10 samples by combining 20.0 μl of 2× Titanium Taq Master Mix (which contained the FRET Cassette). To each well of an APPLIED BIOSYSTEMS 384 PCR plate (Cat #4343814) was added 2.0 μl of 10 ng/μl mouse genomic DNA samples and 2 μl of the reactions mix. Five positive DNA samples and two No Template Controls (which in this case was PCR grade water) were added to the reaction wells of the 384 wellplate. The APPLIED BIOSYSTEMS 7900 Real Time PCR thermocycler conditions were set such that there was a 15 denaturization step at 94° C. The denaturization step was followed by 40 cycles of 94° C. for 20 seconds and 37° C. for one minute. The APPLIED BIOSYSTEMS 7900 was run with 9600 emulation mode. The Real Time PCR plot shown in FIG. 13 was acquired.

FIG. 13 illustrates typical Real-Time PCR or Quantitative PCR data. Each cycle of the PCR reaction is represented on the X-Axis, and the fluorescence is represented on the Y-Axis. The threshold is represented by the green bar intersecting the plots. The Ct data for each sample is collected where the fluorescent amplification plot for each sample intersects the threshold.

Further, if end point analysis is to be done, simple recording of the fluorescence at the last cycle is performed. In this example the 40 cycle was the last cycle.

| Delta Rn | 40 Cycle |
|---|---|
| Positive | 1.5098 |
| Sample | |
| Positive | 1.5281 |
| Sample | |
| Positive | 1.3185 |
| Sample | |
| Positive | 1.2859 |
| Sample | |
| Positive | 1.9361 |
| Sample | |
| Positive | 1.1435 |
| Sample | |
| Negative | 0.0052 |
| Sample | |
| Negative | 0.0375 |
| Sample | |

### Example 7

### Bipartite Probe Quantitative Amplification of Single Copy Eukaryotic (Mouse) DNA Sequence with FRET Oligonucleotides of Equal Length

A Bipartite Probe oligonucleotide with a sequence of:

(SEQ ID NO: 55)
5′-GAAGGTCGGAGTCAACGGATTCTCCCCAGTTCGCTCCA,

a Reverse Primer with a sequence of:

| | |
|---|---|
| 5′-AACGTGAGTGCTAGCGGAGTCTTAA, | (SEQ ID NO: 56) |

a Signal Generation Molecule with a sequence of:

(SEQ ID NO: 57)

5′-Gold 540-atc ggt agc atc gct gaa ggt cgg agt
caa cgg att

5′- and a Quencher Oligonucleotide with a sequence of:

(SEQ ID NO: 58)
5′-aaa aaa aaa aaa aaa aaa aaa agc gat gct acc
gat-BHQ

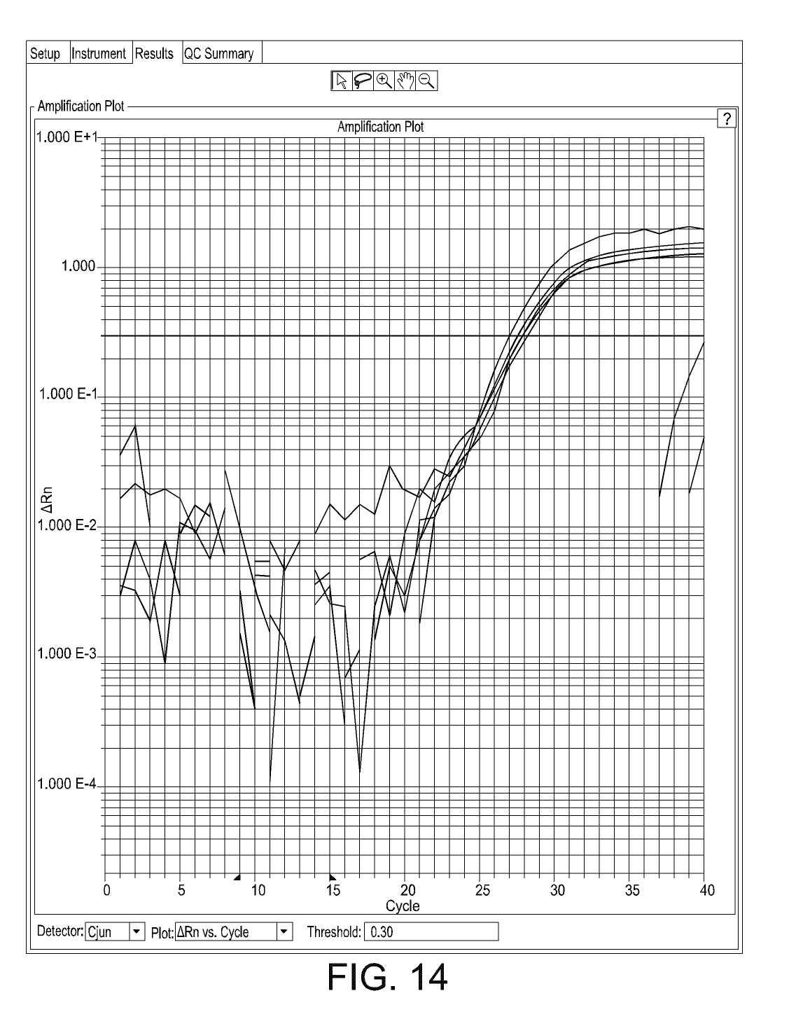

The oligonucleotides were ordered from an oligonucleotide vendor. All of the oligonucleotides were made to 100 μM stock concentrations. Working stocks were made by adding 12.0 μl of Bipartite Probe and 12 μl of Reverse Primer to 76 μl of PCR grade water. Further 1.0 μl of Signal Generation Molecule and 5.0 μl or Quencher Oligonucleotide were added to 1000.0 μl of CLONTECH's Titanium Taq Master Mix (Cat# 639210). A reaction mix was made for 10 samples by combining 20.0 μl of 2× Titanium Taq Master Mix (which contained the FRET Cassette). To each well of an APPLIED BIOSYSTEMS 384 PCR plate (Cat #4343814) was added 2.0 μl of 10 ng/μl mouse genomic DNA samples and 2 μl of the reactions mix. Five positive DNA samples and two No Template Controls (which in this case was PCR grade water) were added to the reaction wells of the 384 wellplate. The APPLIED BIOSYSTEMS 7900 Real Time PCR thermocycler conditions were set such that there was a 15 denaturization step at 94° C. The denaturization step was followed by 40 cycles of 94° C. for 20 seconds and 37° C. for one minute. The APPLIED BIOSYSTEMS 7900 was run with 9600 emulation mode. The Real Time PCR plot shown in FIG. 14 was acquired.

FIG. 14 illustrates typical Real-Time PCR or Quantitative PCR data. Each cycle of the PCR reaction is represented on the X-Axis, and the fluorescence is represented on the Y-Axis. The threshold is represented by the green bar intersecting the plots. The Ct data for each sample is collected where the fluorescent amplification plot for each sample intersects the threshold.

We claim:

1. A method for performing quantitative PCR of a Target Genetic Sequence in a Genetic Sample comprising the steps of:
(1) combining in solution a Bipartite Probe consisting of a target nucleic acid binding sequence capable of hybridizing to a portion of the Target Genetic Sequence with a Nucleic Acid Binding Probe Sequence capable of being transcribed into a Capture Probe Sequence during PCR amplification;
(2) hybridizing a Signal Generation Molecule to the Capture Probe Sequence;
(3) hybridizing a Quencher Oligonucleotide to the non-incorporated Signal Generation Molecule prior to detection at each cycle of PCR; and
(4) generating quantitative PCR Data from each cycle of PCR.

2. The method of claim 1 wherein said Signal Generation Molecule has its fluorescent signal quenched by said Quencher Oligonucleotide at detection temperature of each PCR cycle.

3. The method of claim 2 wherein said detection temperature is about or less than the TM of the Quencher Oligonucleotide.

4. The method of claim 1 wherein said Genetic Sample is genomic DNA.

5. The method of claim 1 wherein said Genetic Sample is Human.

6. The method of claim 1 wherein said Genetic Sample is Mouse.

7. The method of claim 1 wherein said Genetic Sample is Bacterial.

8. The method of claim 1 wherein more than one quantitative reaction is detected in the same reaction.

9. The method of claim 7 wherein a Housekeeping allele reaction is performed simultaneously with another reaction.

10. The method of claim 1 wherein zygosity is determined.

11. The method of claim 1 wherein SNP zygosity is determined.

12. The method of claim 1 wherein said Genetic Sample is eukaryotic.

13. The method of claim 1 wherein said Genetic Sample is prokaryotic.
